(19) United States
(12) Patent Application Publication
(10) Pub. No.: US 2018/0009842 A1
(43) Pub. Date: Jan. 11, 2018

(54) Arginine-rich Peptide Mixture, their Application Thereof in Cervical Cancer Therapy, and a Process for Producing same

(71) Applicants: Dongliang CHEN, Hong Kong (CN); Nascent Peptide Biotechnology Limited, Hong Kong (CN)

(72) Inventors: Dongliang CHEN, HongKong (CN); Yang ZHANG, Wuhan (CN); Lirong HE, Wuhan (CN); Lijian LIU, Wuhan (CN)

(21) Appl. No.: 15/642,278

(22) Filed: Jul. 5, 2017

(30) Foreign Application Priority Data
Jul. 5, 2016 (CN) 201610520561.3

(51) Int. Cl. C07K 1/36; A61K 36/52; C07K 1/14; A61K 38/16; B01D 15/38; B01D 15/32; C07K 1/34; C07K 1/22; C07K 1/18; B01D 15/36; C07K 1/04; A61K 38/17; A61K 38/00

(52) U.S. Cl. CPC C07K 1/36; C07K 1/042; A61K 35/57; A61K 36/52; C07K 1/145; A61K 38/168; B01D 15/3852; A61K 38/1709; B01D 15/325; C07K 1/34; A23L 33/175; C07K 1/22; C07K 1/18; B01D 15/362; A23V 2002/00; A61K 38/00; A61K 2236/13; A61K 2236/15; A61K 2236/53; A61K 2236/39

(57) ABSTRACT

A process for producing an arginine-rich peptide mixture and the application thereof in cervical cancer therapy is provided. The process includes the following steps: A suspension of walnut meal and egg albumin is pretreated with ultrahigh pressure, and then digested by alkaline proteinase and papain in separated steps with the ultrasonic and microwave-assisted extraction. The peptides of interest are isolated from filtration supernatant obtained after the enzyme digestion by reversed phase high-performance liquid chromatography. By using the peptide mixture as a template, acrylic acid and methyl acrylic acid as functional monomers, triethylene glycol dimethacrylate as cross-linking agent, and isopropylthioxanthone in acetone as a photoinitiator, polymerization is induced by ultraviolet light to form a surface imprinted membrane for isolating and enriching the peptides of interest from the supernatant. The arginine content in the peptide mixture is more than 18%. The arginine-rich peptide mixture is able to strongly suppress the proliferation of human cervical cancer Hela cells. The approach is applicable to reduce the cost of production and speed up the commercialization of large-scale production.

### CROSS REFERENCE OF RELATED APPLICATION

This is a non-provisional application that claims the benefit of priority under 35 U.S.C. § 119 to Chinese application number 201610520561.3, filed Jul. 5, 2016, wherein the entire contents of each of which is expressly incorporated herein by reference.

### NOTICE OF COPYRIGHT

A portion of the disclosure of this patent document contains material which is subject to copyright protection. The copyright owner has no objection to any reproduction by anyone of the patent disclosure, as it appears in the United States Patent and Trademark Office patent files or records, but otherwise reserves all copyright rights whatsoever.

### BACKGROUND OF THE PRESENT INVENTION

### Field of Invention

The present invention relates to a process for producing an arginine-rich peptide mixture and the application thereof in cervical cancer therapy, and more particularly, to a process for producing a peptide mixture enriched in arginine by using walnut meal and egg albumin as raw materials and application thereof in cervical cancer therapy.

### Description of Related Arts

Cancer, a general term for malignant tumors, is characterized by body cells losing normal regulation and control, proliferating excessively, having various degrees of differentiation disorder at the same time, and frequently invading adjacent tissues or metastasizing to distant sites. It is a great threat to the health of mankind and a leading cause of excess deaths. “Global Cancer Report”, released by World Health Organization (WHO) in 2014, showed that the number of new cancer cases in China in 2012 was the highest in the world. “Three Year Action Plan to Cancer Prevention and Cure in China (2015-2017)”, jointly released in 2015 by 16 state departments, including National Health and Family Planning Commission and National Development and Reform Commission, clearly indicated that the cancer prevention and cure in our country should focus on 8 types of cancer with the highest morbidity and the greatest harm, among which was cervical cancer, one of the common female cancers.

Conventional therapies for malignant tumors include surgical resection, chemotherapy, radiotherapy, etc. To a certain extent, these processes do work in treatment for cancers, but these treatments often fail in the end once cancer cells spread locally or potentially metastasize. Meanwhile, these conventional therapies will inevitably cause some degree of damage to normal cells and immune system of the body, and lead to adverse reactions, including nausea, loss of appetite, headache and insomnia, hair loss and ulcer, erosion and inflammation, low blood cell count, and etc. Patients often have to discontinue the treatment due to being unable to endure the adverse reactions, which badly impairs quality of life of the cancer patients.

Modern scientific research has shown that many natural bioactive peptides exhibit good efficacy in inhibiting proliferation and metastasis of cancer cells, and have high selectivity and low toxic side effects. These active peptides play a role in the treatment or adjuvant treatment of malignant tumors in a variety of ways, such as inducing tumor differentiation, inhibiting tumor cell growth, inhibiting tumor angiogenesis, increasing the sensitivity of tumor cells to medicines, reducing chemotherapy injury, enhancing the ability of medicines to kill tumor cells, interfering the DNA synthesis of tumor cells, improving body immunity, and so on. In recent years, an increasing number of studies demonstrate that supplemental arginine significantly inhibits tumor cells and induces apoptosis. Polyamines are actively metabolized in tumor cells, and are essential for the rapid division and proliferation of tumor cells. Supplemental arginine can suppress the biosynthesis of polyamines by inhibiting the activity of ornithine decarboxylase, so as to inhibit the proliferation of cancer cells. NO is generated from arginine by nitric oxide synthase. NO is able to block energy metabolism and DNA replication in tumor cells, or lead to DNA damages in tumor cells, resulting in growth inhibition or death of the tumor cells. Moreover, NO is able to down regulate the expression of cellular adhesion molecules and therefore blocks cell adhesion. These adhesion molecules play a key role in the course of tumor cells leaving primary tissues and spreading to other sites to form new lesions. In addition, arginine can promote the production of T lymphocytes and their abilities, increase the generation of interleukin-2 (IL-2) and the expression of the acceptor thereof, regulate and activate macrophages in the body, and therefore improve the tumor host's immunity. There have been studies abroad in which researcher began to try to use amino acid imbalance therapies in cancer patients. In these therapies, the levels of some amino acids in the bodies of tumor hosts were adjusted to interfere with the metabolism and function in tumor cells, and in turn tumor growth was inhibited or tumor cell apoptosis was induced. There are other reports that hydrophilic polypeptides enriched in hydrophilic amino acids (such as arginine) are able to specifically interact with tumor cells through electrostatic attraction, which leads to fast membranolysis, leakage of cellular contents, and finally cell death. There are also studies which alleged that basic amino acids (arginine, lysine, etc.) in the molecular structure of a polypeptide would have some effect on its anti-tumor activity. All of these provide a solid theoretical basis for the anti-cancer efficacy of the present arginine-rich peptide mixture.

Walnut meal is a by-product of walnut processing, with a high protein digestibility. It contains plenty and full range of amino acids. However, it is mainly used in feed industry in our country, without sufficient deep processing, which leads to its depreciation. Egg albumin, which can be thoroughly digested and absorbed in human body, is not only rich in eight essential amino acids, but also has an amino acid composition similar to that of human proteins. For the first time, an animal and plant protein mixture derived from eggs and walnuts is used as a raw material in the present invention to prepare an active peptide mixture enriched in arginine by a series of processes, such as ultrahigh pressure-ultrasonic-microwave assisted enzymatic digestion, surface molecular imprinted membrane isolation and purification. The active peptide mixture can be used in cervical cancer therapy. It provides nutrition to patients and at the same time inhibits the proliferation of cancer cells, so that quality of life in cancer patients is highly improved. Studies related to this application were not reported to date.

### SUMMARY OF THE PRESENT INVENTION

The technical problem to be resolved by the present invention is to provide a process for producing a new arginine-rich peptide mixture and its application in cervical cancer therapy. This peptide mixture is enriched in arginine, and has a strong inhibitory effect on the proliferation of cervical cancer cells.

To resolve the afore-mentioned technical problem, the following technical solution is provided:

A process for producing of an arginine-rich peptide mixture, said process includes the following steps:
(a) Defatted and pulverized walnut meal, egg albumin and water are well mixed and stirred, pretreated with ultrahigh pressure, and subsequently enzyme digested with the assistance of ultrasonic-microwave, wherein the enzymes are inactivated by raising temperature and a supernatant is collected after plate and frame pressure filtration; and
(b) The supernatant is freeze-dried, and the peptides of interest in the freeze-dried coarse powder are subsequently isolated by using reversed phase high-performance liquid chromatography (RP-HPLC): Everest C18 (4.6×250 mm, 5 μm, 238EV54) is used as reversed phase column, acetonitrile-water solution as mobile phase, and trifluoroacetic acid as anionic ion pair reagent; detection is performed at 214 nm wavelength. The column is washed with pure acetonitrile before loading; 25 mg freeze-dried powder is dissolved in the mobile phase with a total volume of 25 mL and filtered through a 0.45 μm microfiltration membrane. Loading volume is 20 μL and column temperature is 30° C. The isolation conditions used are as follows: acetonitrile concentration: 18% (v/v), trifluoroacetic acid concentration: 0.09% (v/v), and flow rate: 1.0 mL/min; 3 eluted fractions, with retention times of 9.64 min, 11.36 min, and 13.80 min, are collected; after freeze-dried, an arginine-rich peptide mixture powder is obtained.

Preferably, said process for producing the arginine-rich peptide mixture further includes a step of isolation and enrichment of the arginine-rich peptide mixture by using a surface imprinted membrane. The step is as follows:
(c) A cover slip and a slide are immersed in Piranha solution (concentrated sulfuric acid and 30% hydrogen peroxide at a volume ratio of 3:1), wherein after ultrasonic cleaning for 1.5˜2.5 h, they are cleaned with pure water and dried with nitrogen before use, wherein the cleaned cover slip is immersed in a solution of the arginine-rich peptide mixture in water to obtain a peptide mixture-immobilized template; the cleaned slide is immersed in a 0.5%˜1.5% (v/v) 3-aminopropyltriethoxysilane (APTES) solution in methanol and shaken at 20˜40 rpm for 15˜45 min. Then, it is rinsed with methanol and dried. A silanized slide is thus obtained. A prepolymer mixture is prepared by well mixing functional monomers acrylic acid (AA) and methyl acrylic acid (MAA) and cross-linking agent triethylene glycol dimethacrylate (TEGDMA) and adding a photoinitiator isopropylthioxanthone (ITX). After purged with nitrogen, the prepolymer mixture is spread on a surface of the silanized slide. Subsequent to rotation, the slide is covered with the peptide mixture-immobilized template. When polymerization, induced with ultraviolet light, is completed, the glass slides are immersed in a solution of 8˜12% (m/v) SDS: 8˜12% (v/v) HAc. The cover slip is removed. After shaken at 80˜160 rpm for 4˜8 h, the slide is rinsed to neutral with pure water under agitation and an arginine-rich peptide mixture surface imprinted membrane is obtained.
(d). The arginine-rich peptide mixture surface imprinted membrane prepared in step (c) is immersed in the supernatant obtained in step (a); after shaken at 20˜40 rpm for 1˜6 h, the imprinted membrane, together with the absorbed peptides of interest, is taken out, and immersed in a 0.5˜1.6 mol/L NaCl solution. At the same time, a 100˜300 W ultrasonic wave is applied for 10˜50 min to assist the elution. NaCl is removed from the collected eluted solution by using cation exchange resin. An arginine-rich peptide mixture powder is obtained after the eluate is being low temperature spray dried. The eluted surface imprinted membrane is immersed in the supernatant obtained in step (a) again after being rinsed with pure water and the subsequent processes are repeated to isolate arginine-rich peptide mixture.
Preferably, in step (a), the defatted and pulverized walnut meal is mixed with egg albumin at a ratio of 3˜6:1 by weight, and the resulting protein meal mixture is well-mixed with water at a weight to volume ratio of 1:4˜14. After stirred for 1.5˜2.5 h at room temperature, the mixture is put into an ultrahigh pressure apparatus with an applied pressure of 200˜600 Mpa for 10˜30 min to obtain an ultrahigh pressure pretreated suspension. The suspension is kept at 40˜60° C. and pH is adjusted to 9˜10. 2%˜6% of alkaline proteinase by weight of the suspension is added and well mixed. In the meantime, ultrasonic-microwave is applied to assist the enzymatic digestion, with an ultrasonic power of 200˜400 W for 10˜20 min and microwave power of 200˜600 W for 5˜15 min. After 1.5˜2.5 h of enzymatic digestion, pH is adjusted to 6˜8; 2%˜6% of papain by weight of the suspension is added and well mixed. At the same time, ultrasonic-microwave is applied to assist the enzymatic digestion, with an ultrasonic power of 200˜400 W for 10˜20 min and a microwave power of 200˜600 W for 5˜15 min. After 2˜3 h of enzymatic digestion, the temperature is raised to inactivate the enzymes and a supernatant is collected following plate and frame pressure filtration.

Preferably, in step (c), the cleaned cover slip is immersed in a 1.5˜10 g/L arginine-rich peptide mixture solution in water and shaken at 20˜40 rpm for 4˜8 h. Then it is rinsed with pure water and dried and a peptide mixture-immobilized template is thus obtained.

Preferably, in step (c), the prepolymer mixture is prepared by mixing the functional monomers acrylic acid (AA) and methyl acrylic acid (MAA) and cross-linking agent triethylene glycol dimethacrylate (TEGDMA) at a volume ratio of 0.5˜3.5:0.5˜2.5:4˜11 and adding 0.2˜0.8 volume of a 1˜4 mmol/L isopropylthioxanthone (ITX) solution in acetone as a photoinitiator.

Preferably, in step (c), after purged with nitrogen for 20˜40 min, the prepolymer mixture is spread on a surface of the silanized slide fixed on a rotator. After the rotator is rotated at 100˜400 rpm for 2˜10 s, the slide is covered with the peptide mixture-immobilized template and polymerization is induced by a 365 nm ultraviolet light and kept for 3˜6 h.

Preferably, in step (d), the absorption-elution circle of the arginine-rich peptide mixture surface imprinted membrane is repeated more than 10 times.

The present invention also provides an arginine-rich peptide mixture, which is prepared by the above processes.

Preferably, arginine content in the peptide mixture is more than 18%.

The applications of the arginine-rich peptide mixture prepared by the above mentioned processes in the preparation of health foods, foods for special dietary uses, ordinary foods and drugs related to cervical cancer therapy are also provided.

Beneficial effects of the present invention are as follows:

When considered in an aspect of efficacy, the arginine-rich peptide mixture prepared by the process of the present invention is able to significantly inhibit the proliferation of cervical cancer cells, and thereby suppress the tumor growth, so as to delay the deterioration of cervical cancer. Malnutrition is a major problem that the cancer patients have to face. The peptide mixture prepared by the present process helps to reduce the absorption inhibition resulted from free amino acids competing for the common absorption sites during the intestinal digestion. A large number of scientific studies have demonstrated that, when a nitrogen source is in a form of peptides, the overall protein accumulation is higher than that through the intake of corresponding amino acids or intact protein. The arginine-rich peptide mixture of the present invention provides nutrition to patients and, at the same time inhibits the proliferation of cancer cells, so that quality of life in cancer patients is effectively improved.
When considered in an aspect of safety, it is provided in GB 29922-2013, National Food Safety Standard—General Principles for the Formula Foods for Special Medical Purpose, that inedible materials derived from the hydrolysis of animals and plants can't be used as a source of free amino acids. The arginine-rich peptide mixture of the present invention is produced from natural food stuff, such as walnuts and eggs, which is consistent with the nutritional concept of nature and health pursued nowadays. Moreover, the enzymes used for hydrolysis are also in accordance with the relevant provisions provided in GB 2760-2014, National Food Safety Standards—Standards for Uses of Food Additives.
When considered in an aspect of preparation, the conditions used in the process of the present invention are mild, and all the instruments involved are the ones commonly used in food and drug industry. The efficiency of the absorption-elution of the surface imprinted membrane is good and the re-utilization rate is high. The present approach is applicable to reduce the cost of production and speed up the commercialization of large-scale production.

Still further objects and advantages will become apparent from a consideration of the ensuing description and drawings.

These and other objectives, features, and advantages of the present invention will become apparent from the following detailed description, the accompanying drawings, and the appended claims.

### BRIEF DESCRIPTION OF THE DRAWINGS

FIG. 1 is a flow diagram showing the preparation of the arginine-rich peptide mixture of the present invention.

FIG. 2 is a HPLC chromatogram of the arginine-rich peptide mixture of the present invention.

FIG. 3 is a diagram showing the preparation of the arginine-rich peptide mixture surface imprinted membrane of the present invention.

FIG. 4 is an atomic force microscope image showing the arginine-rich peptide mixture immobilized template of the present invention.

FIG. 5 is a scanning electron microscope image showing the arginine-rich peptide mixture surface imprinted membrane of the present invention.

FIG. 6 shows the reutilization of the arginine-rich peptide mixture surface imprinted membrane of the present invention.

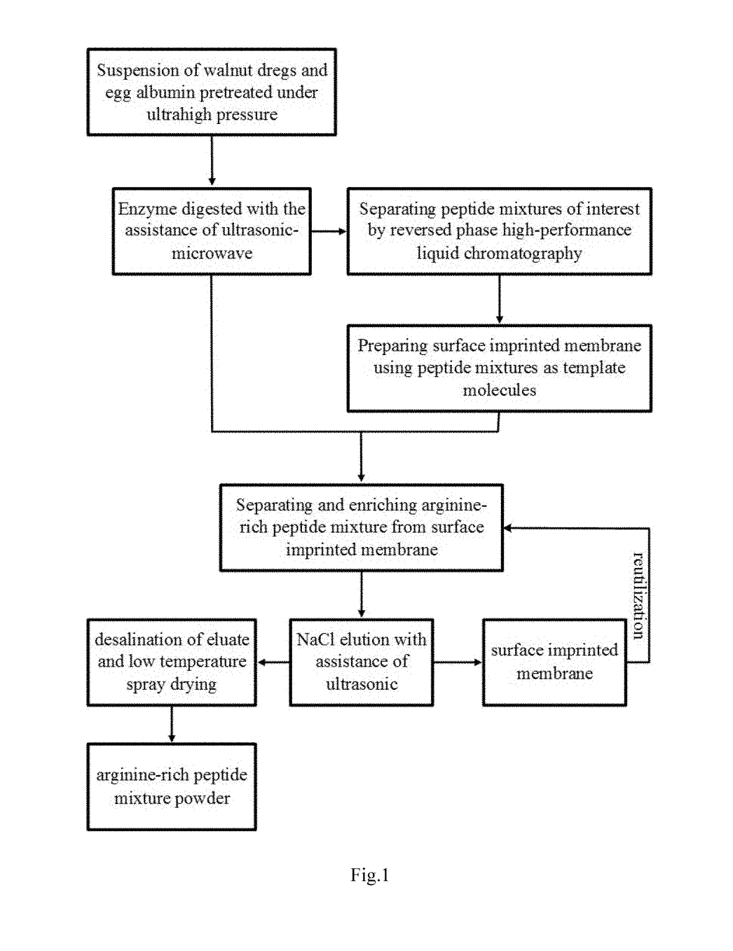

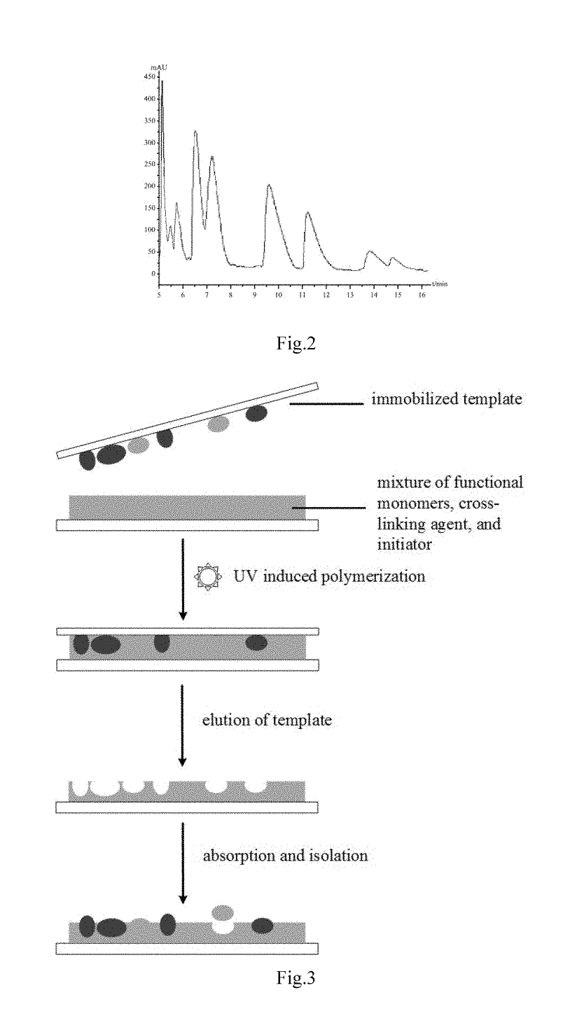

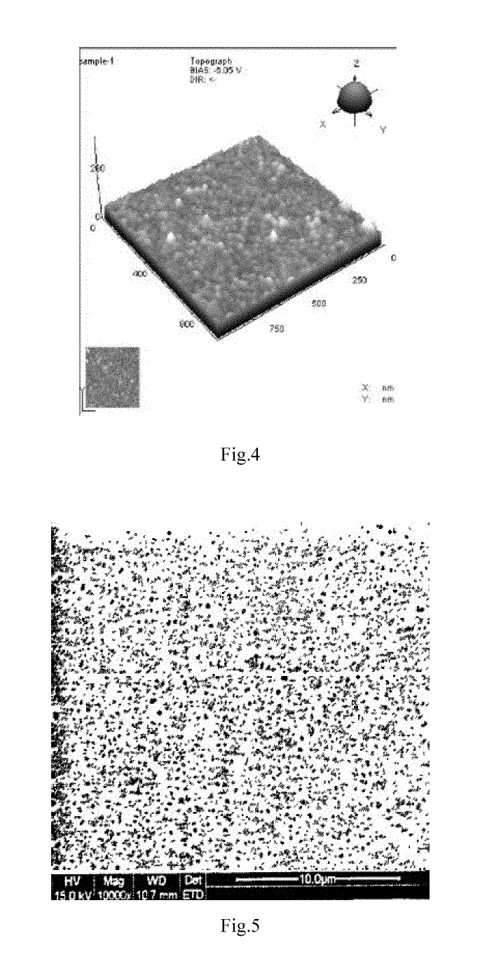

### DETAILED DESCRIPTION OF THE PREFERRED EMBODIMENTS

The following description is disclosed to enable any person skilled in the art to make and use the present invention. Preferred embodiments are provided in the following description only as examples and modifications will be apparent to those skilled in the art. The general principles defined in the following description would be applied to other embodiments, alternatives, modifications, equivalents, and applications without departing from the spirit and scope of the present invention.

The following examples are provided for the purpose of further illustrating the present invention but are in no way to be taken as limiting.

### EXAMPLE 1

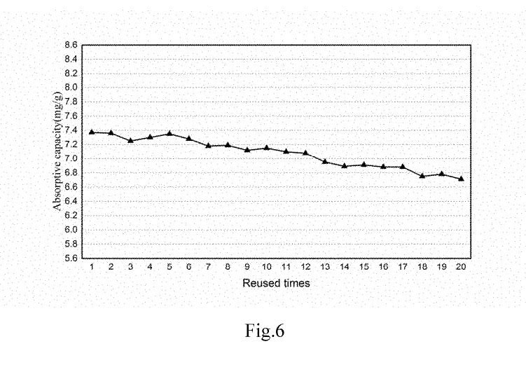

The process for producing the arginine-rich peptide mixture of the present invention includes the following steps:
(a) Defatted and pulverized walnut dregs is mixed with egg albumin at a ratio of 4:1 by weight, and the resulting protein meal mixture is well-mixed with water at a weight to volume ratio of 1:8. After stirred for 2 h at room temperature, it is put into an ultrahigh pressure apparatus with an applied pressure of 400 Mpa for 20 min to obtain an ultrahigh pressure treated suspension. The suspension is kept at 50° C. and pH is adjusted to 9. 3.5% of alkaline proteinase by weight of the suspension is added and well mixed. In the meantime, ultrasonic-microwave is applied to assist the enzymatic digestion, with an ultrasonic power of 300 W for 12 min and a microwave power of 400 W for 8 min. After 2 h of enzymatic digestion, pH is adjusted to 7. 3.5% of papain by weight of the suspension is added and well mixed. At the same time, ultrasonic-microwave is applied to assist the enzymatic digestion, with an ultrasonic power of 300 W for 15 min and a microwave power of 400 W for 10 min. After 2.5 h of enzymatic digestion, the temperature is raised to inactivate the enzymes. Supernatant is collected following plate and frame pressure filtration, in which protein content is 93.6% and peptide content is 88.5%.
(b) After the supernatant is freeze-dried, the peptides of interest in the freeze-dried coarse powder are isolated by using reversed phase high-performance liquid chromatography (RP-HPLC). Everest C18 (4.6×250 mm, 5 μm, 238EV54) is used as reversed phase column, acetonitrile-water solution as mobile phase, and trifluoroacetic acid as anionic ion pair reagent. Detection is performed at 214 nm wavelength. The column is washed with pure acetonitrile before loading. 25 mg freeze-dried powder is dissolved in the mobile phase with a total volume of 25 mL and filtered through a 0.45 μm microfiltration membrane. Loading volume is 20 μL and column temperature is 30° C. The isolation conditions used are as follows: acetonitrile concentration: 18% (v/v) trifluoroacetic acid concentration: 0.09% (v/v), and flow rate: 1.0 mL/min. 3 eluted fractions, with retention times of 9.64 min, 11.36 min and 13.80 min, are collected. After freeze-dried, an arginine-rich peptide mixture powder is obtained, in which the content of peptides of interest is 23.6% of the coarse powder.
(c) Glass slides (a cover slip and a slide) are immersed in Piranha solution (concentrated sulfuric acid and 30% hydrogen peroxide at a volume ratio of 3:1). After ultrasonic cleaning for 2 h, they are cleaned with pure water and dried with nitrogen before use. The cleaned cover slip is immersed in a solution of arginine-rich peptide mixture in water (5 g/L) and shaken at 20 rpm for 6 h. Then it is rinsed with pure water and dried. A peptide mixture-immobilized template is thus obtained. The cleaned slide is immersed in a 1% (v/v) 3-aminopropyltriethoxysilane solution in methanol and shaken at 20 rpm for 30 min. Then it is rinsed with methanol and dried. A silanized slide is thus obtained. A prepolymer mixture is prepared by well mixing functional monomers acrylic acid (AA) and methyl acrylic acid (MAA) and cross-linking agent triethylene glycol dimethacrylate (TEGDMA) at a volume ratio of 2:1:7 and adding 0.5 volume of isopropylthioxanthone (ITX) solution in acetone (2.5 mmol/L) as a photoinitiator. After purged with nitrogen for 30 min, the prepolymer mixture is spread on a surface of the silanized slide fixed on a rotator. After the rotator is rotated at 200 rpm for 4 s, the slide is covered with the peptide mixture-immobilized template. Polymerization is induced by a 365 nm ultraviolet light and kept for 4 h. When the polymerization is completed, the glass slides are immersed in a solution of 10% (m/v) SDS: 10% (v/v) HAc. The cover slip is removed. After shaken at 120 rpm for 6 h, the slide is rinsed to neutral with pure water under agitation. An arginine-rich peptide mixture surface imprinted membrane is obtained.
(d) The arginine-rich peptide mixture surface imprinted membrane prepared in step (c) is immersed in the supernatant obtained in step (a). After shaken at 20 rpm for 4 h, the imprinted membrane, together with the absorbed peptides of interest, is taken out, and immersed in a 1 mol/L NaCl solution. At the same time, a 200 W ultrasonic wave is applied for 30 min to assist the elution. NaCl is removed from the eluted solution by using cation exchange resin. An arginine-rich peptide mixture powder is obtained after the eluate is low temperature spray dried. The adsorption rate of the surface imprinted membrane is 80.5% (see Table 1). The arginine content in the spray dried peptide mixture is 21.3% (see Table 2). The eluted surface imprinted membrane is immersed in the supernatant obtained in step (a) again after being rinsed with pure water. The subsequent processes are repeated to isolate the arginine-rich peptide mixture. It is reused 20 times. The regeneration rate is 91.1% (see FIG. 6).
(e) The adsorption rate of the surface imprinted membrane for the peptides of interest in the supernatant obtained after the plate and frame pressure filtration is determined by high performance liquid chromatography in triplicate. The equation is: adsorption rate (%)=content of peptides of interest in the eluate/content of peptides of interest in the supernatant×100%. The result is showed in Table 1:

TABLE 1
Adsorption rate of the surface imprinted membrane
for the arginine-rich peptide mixture

| | content of peptides of | content of peptides of | |
|---|---|---|---|
| | interest in the | interest in the | adsorption |
| | eluate(mg/L) | supernatant(mg/L) | rate(%) |
| 1 | 67.6 | 83.0 | 81.4 |
| 2 | 68.4 | 84.3 | 81.1 |
| 3 | 66.5 | 82.6 | 80.5 |

The arginine-rich peptide mixture surface imprinted membrane is subjected to the absorption-elution-reabsorption circle. The adsorptive capacity is reduced from the original 7.38 mg/g to 6.72 mg/g after reused 20 times. The regeneration rate is as high as 91.1%, which shows an excellent reutilization property.
(f) After acidic hydrolysis, the contents of all kinds of amino acid in the arginine-rich peptide mixture are detected using amino acid analyzer. The results are shown in Table 2:

TABLE 2
Amino acid analysis results of the arginine-rich peptide mixture

| Amino acid | | Content mg/100 mg |
|---|---|---|
| aspartic acid | Asp | 6.3 |
| threonine | Thr | 3.0 |
| Serine | Ser | 5.5 |
| glutamic acid | Glu | 12.2 |
| glycine | Gly | 3.3 |
| alanine | Ala | 3.6 |
| cysteine | Cys | 1.0 |
| Valine | Val | 3.4 |
| methionine | Met | 2.7 |
| isoleucine | Ile | 3.2 |
| leucine | Leu | 5.1 |
| tyrosine | Tyr | 2.6 |
| phenylalanine | Phe | 3.9 |
| histidine | His | 1.5 |
| Lysine | Lys | 3.7 |
| arginine | Arg | 21.3 |
| proline | Pro | 3.1 |

Note:
Tryptophan, asparagine and glutamine are destroyed in the acidic hydrolysis.

### EXAMPLE 2

The process for producing the peptide mixture of the present invention includes the following steps:
(a) Defatted and pulverized walnut dregs is mixed with egg albumin at a ratio of 3:1 by weight, and the resulting protein dregs mixture is well-mixed with water at a weight to volume ratio of 1:6. After stirred for 1.5 h at room temperature, it is put into an ultrahigh pressure apparatus with an applied pressure of 300 Mpa for 15 min to obtain an ultrahigh pressure pretreated suspension. The suspension is kept at 50° C. and pH is adjusted to 9. 3% of alkaline proteinase by weight of the suspension is added and well mixed. In the meantime, ultrasonic-microwave is applied to assist the enzymatic digestion, with an ultrasonic power of 200 W for 15 min and a microwave power of 300 W for 10 min. After 1.5 h of enzymatic digestion, pH is adjusted to 7. 3% of papain by weight of the suspension is added and well mixed. At the same time, ultrasonic-microwave is applied to assist the enzymatic digestion, with an ultrasonic power of 200 W for 18 min and a microwave power of 300 W for 12 min. After 2 h of enzymatic digestion, the temperature is raised to inactivate the enzymes. A supernatant is collected following plate and frame pressure filtration, in which protein content is 90.3% and peptide content is 81.7%.
(b) After the supernatant is freeze-dried, the peptides of interest in the freeze-dried coarse powder are isolated by using reversed phase high-performance liquid chromatography (RP-HPLC). Everest C18 (4.6×250 mm, 5 μm, 238EV54) is used as reversed phase column, acetonitrile-water solution as mobile phase, and trifluoroacetic acid as anionic ion pair reagent. Detection is performed at 214 nm wavelength. The column is washed with pure acetonitrile before loading. 25 mg freeze-dried powder is dissolved in the mobile phase with a total volume of 25 mL and filtered through a 0.45 microfiltration membrane. Loading volume is 20 μL and column temperature is 30° C. The isolation conditions used are as follows: acetonitrile concentration: 18% (v/v), trifluoroacetic acid concentration: 0.09% (v/v), and flow rate: 1.0 mL/min. 3 eluted fractions, with retention times of 9.64 min, 11.36 min and 13.80 min, are collected. After freeze-dried, an arginine-rich peptide mixture powder is obtained, in which the content of peptides of interest is 19.8% of the coarse powder.
(c) Glass slides (a cover slip and a slide) are immersed in Piranha solution (concentrated sulfuric acid and 30% hydrogen peroxide at a volume ratio of 3:1). After ultrasonic cleaning for 1.5 h, they are cleaned with pure water and dried with nitrogen before use. The cleaned cover slip is immersed in a solution of arginine-rich peptide mixture in water (3 g/L) and shaken at 30 rpm for 4 h. Then it is rinsed with pure water and dried. A peptide mixture-immobilized template is thus obtained. The cleaned slide is immersed in a 0.8% (v/v) 3-aminopropyltriethoxysilane solution in methanol and shaken at 30 rpm for 18 min. Then it is rinsed with methanol and dried. A silanized slide is thus obtained. A prepolymer mixture is prepared by well mixing functional monomers acrylic acid (AA) and methyl acrylic acid (MAA) and cross-linking agent triethylene glycol dimethacrylate (TEGDMA) at a volume ratio of 1.5:0.8:7 and adding 0.3 volume of isopropylthioxanthone (ITX) solution in acetone (3 mmol/L) as a photoinitiator. After purged with nitrogen for 20 min, the prepolymer mixture is spread on a surface of the silanized slide fixed on a rotator. After the rotator is rotated at 100 rpm for 6 s, the slide is covered with the peptide mixture-immobilized template. Polymerization is induced by a 365 nm ultraviolet light and kept for 5 h. When the polymerization is completed, the glass slides are immersed in a solution of 8% (m/v) SDS: 8% (v/v) HAc. The cover slip is removed. After shaken at 160 rpm for 4 h, the slide is rinsed to neutral with pure water under agitation. An arginine-rich peptide mixture surface imprinted membrane is obtained.
(d) The arginine-rich peptide mixture surface imprinted membrane prepared in step (c) is immersed in the supernatant obtained in step (a). After shaken at 30 rpm for 5 h, the imprinted membrane, together with the absorbed peptides of interest, is taken out, and immersed in 0.8 mol/L NaCl solution. At the same time, a 100 W ultrasonic wave is applied for 40 min to assist the elution. NaCl is removed from the eluted solution by using cation exchange resin. An arginine-rich peptide mixture powder is obtained after the eluate is low temperature spray dried. The adsorption rate of the surface imprinted membrane is 71.3% and the regeneration rate is 84.2% after reused for 20 times. The arginine content in the spray dried peptide mixture is 18.9%.

### EXAMPLE 3

The process for producing the peptide mixture of the present invention includes the following steps:
(a) Defatted and pulverized walnut dregs particles are mixed with egg albumin at a ratio of 5:1 by weight, and the resulting protein dregs mixture is well-mixed with water at a weight to volume ratio of 1:10. After stirred for 2.5 h at room temperature, it is put into an ultrahigh pressure apparatus with an applied pressure of 500 Mpa for 25 min to obtain an ultrahigh pressure pretreated suspension. The suspension is kept at 50° C. and pH is adjusted to 9. 4% of alkaline proteinase by weight of the suspension is added and well mixed. In the meantime, ultrasonic-microwave is applied to assist the enzymatic digestion, with an ultrasonic power of 400 W for 20 min and a microwave power of 500 W for 5 min. After 2.5 h of enzymatic digestion, pH is adjusted to 7. 4% of papain by weight of the suspension is added and well mixed. At the same time, ultrasonic-microwave is applied to assist the enzymatic digestion, with an ultrasonic power of 400 W for 20 min and a microwave power of 500 W for 8 min. After 3 h of enzymatic digestion, the temperature is raised to inactivate the enzymes. A supernatant is collected following plate and frame pressure filtration, in which protein content is 92.1% and peptide content is 84.7%.
(b) After the supernatant is freeze-dried, the peptides of interest in the freeze-dried coarse powder are isolated by using reversed phase high-performance liquid chromatography (RP-HPLC). Everest C18 (4.6×250 mm, 5 μm, 238EV54) is used as reversed phase column, acetonitrile-water solution as mobile phase, and trifluoroacetic acid as anionic ion pair reagent. Detection is performed at 214 nm wavelength. The column is washed with pure acetonitrile before loading. 25 mg freeze-dried powder is dissolved in the mobile phase with a total volume of 25 mL and filtered through a 0.45 μm microfiltration membrane. Loading volume is 20 μL and column temperature is 30° C. The isolation conditions used are as follows: acetonitrile concentration: 18% (v/v), trifluoroacetic acid concentration: 0.09% (v/v), and flow rate: 1.0 mL/min. 3 eluted fractions, with retention times of 9.64 min, 11.36 min, and 13.80 min, are collected. After freeze-dried, an arginine-rich peptide mixture powder is obtained, in which the content of peptides of interest is 21.9% of the coarse powder.
(c) Glass slides (a cover slips and a slide) are immersed in Piranha solution (concentrated sulfuric acid and 30% hydrogen peroxide at a volume ratio of 3:1). After ultrasonic cleaning for 2.5 h, they are cleaned with pure water and dried with nitrogen before use. The cleaned cover slip is immersed in a solution of arginine-rich peptide mixture in water (4 g/L) and shaken at 40 rpm for 8 h. Then, it is rinsed with pure water and dried. A peptide mixture-immobilized template is thus obtained. The cleaned slide is immersed in a 1.2% (v/v) 3-aminopropyltriethoxysilane solution in methanol and shaken at 40 rpm for 40 min. Then it is rinsed with methanol and dried. A silanized slide is thus obtained. A prepolymer mixture is prepared by well mixing functional monomers acrylic acid (AA) and methyl acrylic acid (MAA) and cross-linking agent triethylene glycol dimethacrylate (TEGDMA) at a volume ratio of 3:1.5:8 and adding 0.6 volume of isopropylthioxanthone (ITX) solution in acetone (4 mmol/L) as a photoinitiator. After purged with nitrogen for 40 min, the prepolymer mixture is spread on a surface of the silanized slide fixed on a rotator. After the rotator is rotated at 300 rpm for 3 s, the slide is covered with the peptide mixture-immobilized template. Polymerization is induced by a 365 nm ultraviolet light and kept for 6 h. When the polymerization is completed, the glass slides are immersed in a solution of 10% (m/v) SDS: 8% (v/v) HAc. The cover slip is removed. After shaken at 140 rpm for 8 h, the slide is rinsed to neutral with pure water under agitation. An arginine-rich peptide mixture surface imprinted membrane is obtained.
(d). The arginine-rich peptide mixture surface imprinted membrane prepared in step (c) is immersed in the supernatant obtained in step (a). After shaken at 40 rpm for 6 h, the imprinted membrane, together with the absorbed peptides of interest, is taken out, and immersed in a 1.2 mol/L NaCl solution. At the same time, a 300 W ultrasonic wave is applied for 20 min to assist the elution. NaCl is removed from the eluted solution by using cation exchange resin. An arginine-rich peptide mixture powder is obtained after the eluate is low temperature spray dried. The adsorption rate of the surface imprinted membrane is 77.5% and the regeneration rate is 88.0% after reused 20 times. The arginine content in the spray dried peptide mixture is 20.4%.

### EXAMPLE 4

### Inhibition of Human Cervical Cancer Cell Proliferation by the Arginine-Rich Peptide Mixture is Evaluated Using MTT Method

### 1. Instruments and Materials

METERTIECHΣ960 microplate reader is a product from METERTECH Inc., Taiwan; (MCO-15AC) CO₂ cell incubator is a product from Sanyo Co., Ltd, Japan; 1300SERIES A2 biosafety cabinet is a product from Thermo Fisher Scientific, USA; TDL-50B low speed benchtop centrifuge is a product from Shanghai Anting Scientific Instruments Factory; and model D-1 automatic steam sterilization pot is a product from Beijing Faen Technology & Trade Co. Ltd.

Human cervical cancer Hela cells are purchased from Cell Bank of Chinese Academy of Sciences; RPMI-1640 medium is purchased from GIBCO, USA; Fetal bovine serum is purchased from Tianjin Chuanye Biochemical Products Co. Ltd.; and the arginine-rich peptide mixture is the one prepared in Example 1.

### 2. Experimental Method

Human cervical cancer Hela cells in exponential growth phase are collected, formulated as a single-cell suspension with a concentration of 3×10⁴ cells/mL, and inoculated in a 96-well plate (100 μL/well). After 24 h of growth in the incubator at 37° C., under 5% CO₂, cell adherence occurs and the culture medium is discarded. Control group and experimental group are established: different concentrations of the arginine-rich peptide mixture prepared in Example 1 are added to the experimental group (4 mg/mL, 6 mg/mL or 8 mg/mL, diluted with RPMI-1640 medium) and the same volumes of RPMI-1640 medium are added to the control group. After 24 h of growth, MTT working solution (5 mg/mL) is added in 10 μL/well. 4 h later, 100 μL supernatant is removed from the top of the culture medium and 100 μL formazan solubilization solution is added. After another 4 h of incubation, the absorbance is detected with microplate reader (wavelength 570 nm). The inhibition rate of the arginine-rich peptide mixture on cell proliferation is calculated. The determination is performed in quadruplicate wells in each group.

Inhibition rate (%)=(control group A₅₇₀−experimental group A₅₇₀)/control group A₅₇₀×100%

### 3. Experimental Results

It is indicated in Table 3 that 3 different concentrations (4 mg/mL, 6 mg/mL and 8 mg/mL) of the arginine-rich peptide mixture significantly inhibit the proliferation of Hela cells. Moreover, the inhibition rate is increased with the increase of concentration of the arginine-rich peptide mixture.

TABLE 3
Inhibition rates of the arginine-rich peptide mixture on the proliferation of
human cervical cancer Hela cells (n = 4, x̅ ± s)

| Items | Inhibition rates (%) |
|---|---|
| Control group | — |
| 4 mg/mL | 30.16 ± 0.47 |
| 6 mg/mL | 68.74 ± 0.55 |
| 8 mg/mL | 72.75 ± 0.32 |

### 4. Experimental Conclusions

The arginine-rich peptide mixture of the present invention has a strong inhibitory activity on the proliferation of cervical cancer cells and the inhibition is in a dose-dependent manner over a certain concentration range.

One skilled in the art will understand that the embodiment of the present invention as shown in the drawings and described above is exemplary only and not intended to be limiting.

It will thus be seen that the objects of the present invention have been fully and effectively accomplished. The embodiments have been shown and described for the purposes of illustrating the functional and structural principles of the present invention and is subject to change without departure from such principles. Therefore, this invention includes all modifications encompassed within the spirit and scope of the following claims.

What is claimed is:

1. A process for producing an arginine-rich peptide mixture, comprising the following steps:
(a) collecting a supernatant comprising peptides of interest, wherein de-fatted and pulverized walnut meal, egg albumin and water are mixed and stirred, pretreated with ultrahigh pressure, and subsequently enzymatic digested with ultrasonic-microwave-assisted extraction, wherein enzymes are inactivated by raising temperature and the supernatant is collected through plate and frame pressure filtration; and
(b) isolating the peptides of interest from the supernatant, wherein the supernatant is freeze-dried, and the peptides of interest in freeze-dried coarse powder are subsequently isolated by using reversed phase high-performance liquid chromatography (RP-HPLC), wherein Everest C18 (4.6×250 mm, 5 μm, 238EV54) is used as reversed phase column, acetonitrile-water solution as mobile phase, and trifluoroacetic acid as anionic ion pair reagent, wherein detection is performed at 214 nm wavelength, wherein column is washed with pure acetonitrile before loading, wherein 25 mg freeze-dried powder is dissolved in mobile phase with a total volume of 25 mL and filtered through a 0.45 μm microfiltration membrane, wherein loading volume is 20 μL and column temperature is 30° C., wherein isolation conditions used include acetonitrile concentration: 18% (v/v), trifluoroacetic acid concentration: 0.09% (v/v), and flow rate: 1.0 mL/min, wherein 3 eluted fractions, with retention times of 9.64 min, 11.36 min, and 13.80 min, are collected, and after freeze-dried, an arginine-rich peptide mixture in powder form is obtained.

2. The process according to claim 1, further comprising a step of isolating and enriching the arginine-rich peptide mixture by using a surface imprinted membrane, which comprises the steps of:
(c) cleaning a cover slip and a slide, wherein the cover slip and the slide are immersed in Piranha solution (Sulfuric acid and 30% Hydrogen peroxide 3:1 (v/v)), wherein after ultrasonic cleaning for 1.5 h to 2.5 h, the cover slip and the slide are cleaned with pure water and dried with nitrogen before use;
producing a peptide mixture-immobilized template by immersing the cleaned cover slip in an aqueous solution of the arginine-rich peptide mixture;
producing a silanized slide, wherein the cleaned slide is immersed in a 0.5% to 1.5% (v/v) 3-aminopropyltriethoxysilane (APTES) solution in methanol and shaken at 20 rpm to 40 rpm for 15 min to 45 min and then rinsed with methanol and dried; and
producing the surface imprinted membrane, wherein a prepolymer mixture is prepared by mixing functional monomers acrylic acid (AA) and methyl acrylic acid (MAA) and cross-linking agent triethylene glycol dimethacrylate (TEGDMA) and adding a photoinitiator isopropylthioxanthone (ITX), wherein after purged with nitrogen, the prepolymer mixture is spread on a surface of the silanized slide, wherein subsequent to rotation, the slide is covered with the peptide mixture-immobilized template, wherein when polymerization, induced with ultraviolet light, is completed, the glass slides are immersed in a solution of 8% to 12% (m/v) SDS: 8% to 12% (v/v) HAc, wherein the cover slip is removed, wherein after shaken at 80 rpm to 160 rpm for 4 h to 8 h, the slide is rinsed to neutral with pure water under agitation, and an arginine-rich peptide mixture surface imprinted membrane is thus obtained; and
(d) isolating and enriching the arginine-rich peptide mixture from the supernatant of the step (a) by using the surface imprinted membrane prepared in the step (c), wherein the arginine-rich peptide mixture surface imprinted membrane is immersed in the supernatant obtained in the step (a), wherein after shaken at 20 rpm to 40 rpm for 1 h to 6 h, the imprinted membrane, together with the absorbed peptides of interest, is taken out and immersed in a 0.5 mol/L to 1.6 mol/L NaCl solution, wherein, at the same time, a 100 W to 300 W ultrasonic wave is applied for 10 min to 50 min to assist the elution, wherein NaCl is removed from the collected eluted solution by using cation exchange resin, wherein an arginine-rich peptide mixture powder is obtained after the eluate solution is low temperature spray dried, wherein the eluted surface imprinted membrane is immersed in the supernatant obtained in the step (a) again after being rinsed with pure water and the subsequent processes are repeated to isolate the arginine-rich peptide mixture.

3. The process according to claim 1, wherein the defatted and pulverized walnut meal is mixed with egg albumin at a ratio of 3 to 6:1 by weight, and the resulting protein dregs mixture is mixed with water at a weight to volume ratio of 1:4 to 14, wherein after stirred for 1.5 h to 2.5 h at room temperature, the mixture is put into an ultrahigh pressure apparatus with an applied pressure of 200 Mpa to 600 Mpa for 10 min to 30 min to obtain an ultrahigh pressure pretreated suspension, wherein the suspension is kept at 40° C. to 60° C. and pH is adjusted to 9 to 10; 2% to 6% of alkaline proteinase by weight of the suspension is added and mixed, wherein, in the meantime, ultrasonic-microwave is applied to assist the enzymatic digestion, with an ultrasonic power of 200 W to 400 W for 10 min to 20 min and a microwave power of 200 W to 600 W for 5 min to 15 min, wherein after 1.5 h to 2.5 h of enzymatic digestion, pH is adjusted to 6 to 8; 2% to 6% of papain by weight of the suspension is added and mixed, wherein, at the same time, ultrasonic-microwave is applied to assist the enzymatic digestion, with an ultrasonic power of 200 W to 400 W for 10 min to 20 min and a microwave power of 200 W to 600 W for 5 min to 15 min, wherein after 2 h to 3 h of enzymatic digestion, the temperature is raised to inactivate the enzymes and the supernatant is collected following plate and frame pressure filtration.

4. The process according to claim 2, wherein, in the step (c), the cleaned cover slip is immersed in a 1.5 g/L to 10 g/L arginine-rich peptide mixture solution in water and shaken at 20 rpm to 40 rpm for 4 h to 8 h, and then it is rinsed with pure water and dried and the peptide mixture-immobilized template is thus obtained.

5. The process according to claim 2, wherein, in the step (c), the prepolymer mixture is prepared by mixing the functional monomers acrylic acid (AA) and methyl acrylic acid (MAA) and cross-linking agent triethylene glycol dimethacrylate (TEGDMA) at a volume ratio of 0.5 to 3.5:0.5 to 2.5:4 to 11 and adding 0.2 to 0.8 volume of a 1 mmol/L to 4 mmol/L isopropylthioxanthone (ITX) solution in acetone as the photoinitiator.

6. The process according to claim 2, wherein, in the step (c), after purged with nitrogen for 20 min to 40 min, the prepolymer mixture is spread on the surface of the silanized slide fixed on a rotator, wherein after the rotator is rotated at 100 rpm to 400 rpm for 2 s to 10 s, the slide is covered with the peptide mixture-immobilized template and polymerization is induced by a 365 nm ultraviolet light and kept for 3 h to 6 h.

7. The process according to claim 2, wherein, in the step (d), the absorption-elution circle of the arginine-rich peptide mixture surface imprinted membrane is repeated more than 10 times.

8. The arginine-rich peptide mixture according to claim 1, wherein arginine content in the peptide mixture is more than 18%.

9. The arginine-rich peptide mixture according to claim 2, wherein arginine content in the peptide mixture is more than 18%.

10. The arginine-rich peptide mixture according to claim 1, wherein the arginine-rich peptide mixture is used in a preparation of health foods, foods for special dietary uses, ordinary foods and drugs related to cervical cancer therapy.

11. The arginine-rich peptide mixture according to claim 2, wherein the arginine-rich peptide mixture is used in a preparation of health foods, foods for special dietary uses, ordinary foods and drugs related to cervical cancer therapy.
